(19) United States
(12) Patent Application Publication
(10) Pub. No.: US 2020/0281757 A1
(43) Pub. Date: Sep. 10, 2020

(54) ELECTRONIC INFRARED PENIS PUMP

(71) Applicant: Bryan DeVore, Snoqualmie, WA (US)

(72) Inventor: Bryan DeVore, Snoqualmie, WA (US)

(21) Appl. No.: 16/291,350

(22) Filed: Mar. 4, 2019

(51) Int. Cl. A61F 5/41; A61N 5/06

(52) U.S. Cl. CPC A61F 5/41; A61N 5/0625; A61N 2005/0659; A61N 2005/0645; A61N 2005/0652; A61F 2005/412

(57) ABSTRACT

Disclosed embodiments provide a penis pump incorporating near infrared light. The penis pump includes a control unit, wherein the control unit includes a power source, and wherein the control unit includes a suction mechanism that is powered by the power source. The penis pump further includes a tube section, coupled to the control unit, configured to receive a human penis, a plurality of light sources housed within the tube section, wherein the plurality of light sources are configured to direct near infrared light onto the human penis when the human penis is inserted into the tube section, and a base section configured to provide a seal around the human penis when the human penis is inserted into the tube section.

### FIELD OF THE EMBODIMENTS

The field of the embodiments of this invention relates generally to vacuum constriction devices and, more particularly, to an infrared vacuum constriction device (aka a “penis pump”).

### BACKGROUND OF THE EMBODIMENTS

Vacuum Constriction Devices (aka “VCDs” or “penis pumps”) involve using a vacuum chamber to expand the corpus cavernosum of the human penis. This promotes penis enlargement and helps to increase both penis length and penis girth (thickness). These devices are further beneficial in that they help to treat erectile dysfunction and increase blood flow. The VCD works by applying constant vacuum force on the penis, which ultimately expands the blood chambers and vascular system in the penis, thus promoting penis enlargement.

### SUMMARY OF THE EMBODIMENTS

According to an aspect of the present invention, a penis pump incorporating near infrared light is provided. The penis pump includes a control unit, wherein the control unit includes a power source, and wherein the control unit includes a suction mechanism that is powered by the power source. The penis pump further includes a tube section, coupled to the control unit, configured to receive a human penis, a plurality of light sources housed within the tube section, wherein the plurality of light sources are configured to direct near infrared light onto the human penis when the human penis is inserted into the tube section, and a base section configured to provide a seal around the human penis when the human penis is inserted into the tube section.

It is an object of the present invention to provide the penis pump incorporating near infrared light, wherein the tube section includes a transparent tube, configured to enable visual inspection of the human penis while the human penis is inserted into the transparent tube.

It is an object of the present invention to provide the penis pump incorporating near infrared light, wherein the plurality of light sources are positioned along a length of the tube section.

It is an object of the present invention to provide the penis pump incorporating near infrared light, wherein the tube section further includes a ruler section positioned along a length of the tube section.

It is an object of the present invention to provide the penis pump incorporating near infrared light, wherein the plurality of light sources are coupled to the ruler section.

It is an object of the present invention to provide the penis pump incorporating near infrared light, wherein the control unit is removably coupled to the tube section.

It is an object of the present invention to provide the penis pump incorporating near infrared light, wherein the power source is a battery.

It is an object of the present invention to provide the penis pump incorporating near infrared light, wherein the power source is an external power source.

It is an object of the present invention to provide the penis pump incorporating near infrared light, wherein the control unit further includes a control panel.

It is an object of the present invention to provide the penis pump incorporating near infrared light, wherein the control panel includes a plurality of buttons.

It is an object of the present invention to provide the penis pump incorporating near infrared light, wherein the plurality of buttons are configured to perform functions selected from the group consisting of: powering on the penis pump; powering off the penis pump; increasing pressure within the penis pump; and decreasing pressure within the penis pump.

It is an object of the present invention to provide the penis pump incorporating near infrared light, wherein the control unit further includes a display configured to display information pertaining to functionalities of the penis pump.

It is an object of the present invention to provide the penis pump incorporating near infrared light, wherein the base section includes silicone.

It is an object of the present invention to provide the penis pump incorporating near infrared light, wherein the penis pump further includes a penis strap configured to prevent testicles from rising into the penis pump during use.

It is an object of the present invention to provide the penis pump incorporating near infrared light, wherein the near infrared light ranges in wavelength from 630 nanometers to 980 nanometers.

### BRIEF DESCRIPTION OF THE DRAWINGS

The structure, operation, and advantages of disclosed embodiments will become further apparent upon consideration of the following description taken in conjunction with the accompanying figures (FIGs.). The figures are intended to be illustrative, not limiting. Certain elements in some of the figures may be omitted, or illustrated not-to-scale, for illustrative clarity.

FIG. 1 shows an infrared penis pump system, according to an embodiment of the present invention.

FIGS. 2A-2D show detail of a power converter, according to an embodiment of the present invention. FIG. 2A is a top view. FIG. 2B is a perspective view. FIG. 2C is a side view. FIG. 2D is a front view.

FIGS. 3A-3D show detail of a removable control unit, according to an embodiment of the present invention. FIG. 3A is a top view. FIG. 3B is a perspective view. FIG. 3C is a side view. FIG. 3D is a front view.

FIGS. 4A-4D show detail of a penis strap, according to an embodiment of the present invention. FIG. 4A is a side view. FIG. 4B is a front view. FIG. 4C is a perspective view. FIG. 4D is a top view.

FIGS. 5A-5D show detail of a base portion, according to an embodiment of the present invention. FIG. 5A is a front view. FIG. 5B is a side view. FIG. 5C is a perspective view. FIG. 5D is a top view.

FIGS. 6A-6D show detail of a tube section, according to an embodiment of the present invention. FIG. 6A is a front view of a ruler section portion of the tube section having multiple light sources. FIG. 6B is a side view of the tube section. FIG. 6C is a front view of the tube section. FIG. 6D is a bottom view of the tube section.

FIGS. 7A-7D show detail of a plug connection, according to an embodiment of the present invention. FIG. 7A is a top view. FIG. 7B is a perspective view. FIG. 7C is a side view. FIG. 7D is a rear view.

### DETAILED DESCRIPTION

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Disclosed embodiments provide an infrared penis pump for promoting enlargement of a human penis. Human bodies have cellular receptors for near infrared light. These cellular receptors, referred to as chromophores, absorb specific wavelengths of near infrared light and use that energy to enhance the production of adenosine triphosphate (ATP) in human cells.

In a process called cellular respiration, chemical energy is converted into chemical energy that the cell can use and stores it in molecules of ATP. With the addition of near infrared radiation energy, the result is more cell power to produce proteins and heal tissue. In multiple studies, near infrared light causes cell tissue to repair itself faster and more efficiently. Near infrared light also enhances the cells' production of nitric oxide, leading to enhanced vasodilation and circulation which is critical to erection firmness. Furthermore, deoxyribonucleic acid (DNA) synthesis in fibroblasts and muscle cells has been quintupled by NASA using light emitted from light emitting diodes (LEDs) alone, in a single application combining 680, 730, and 880 nanometer wavelengths, each at 4 Joules per centimeter squared.

Disclosed embodiments combine penis traction with infrared light, thereby reducing the time required in order to see noticeable improvements and enabling a higher success rate for traction apparatus users.

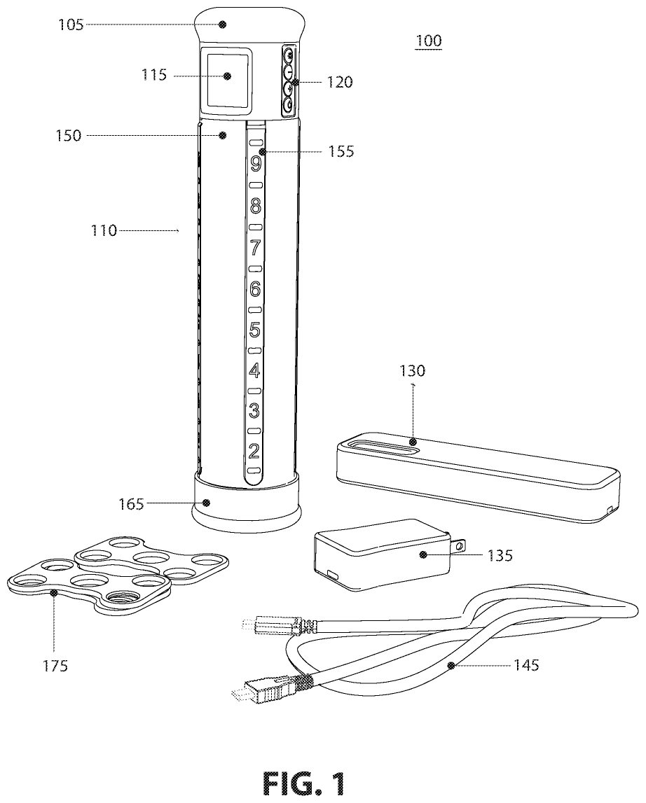

Referring now to FIG. 1, an infrared penis pump system 100 is illustratively depicted, in accordance with an embodiment of the present invention.

According to an embodiment, the infrared penis pump system includes a battery powered removable control unit 105 with Type C power adapter coupled to a tube section 110 configured to enable the insertion of a human penis. It is noted, however, that other suitable types of power adapter and/or any suitable power source may be used in conjunction with the system, while maintaining the spirit of the present invention.

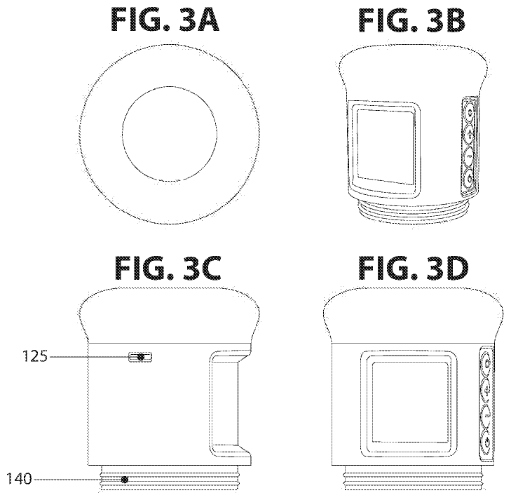

According to an embodiment, the removable control unit 105 (shown various angles in FIGS. 3A-3D) includes a display 115 configured to display information such as, but not limited to, battery levels, system status, pressure status, time duration of use, mode of operation, power indication, and/or any other suitable information. According to an embodiment, the control unit 105 further includes one or more control buttons 120. The control buttons 120 may include power on/off buttons, pressure control (increase/decrease) buttons, gradual pressure release buttons, total release buttons, mode of operation buttons, and/or any other buttons for any suitable functions. FIGS. 3A-3D show detail of the removable control unit 105. FIG. 3A is a top view. FIG. 3B is a perspective view. FIG. 3C is a side view. FIG. 3D is a front view.

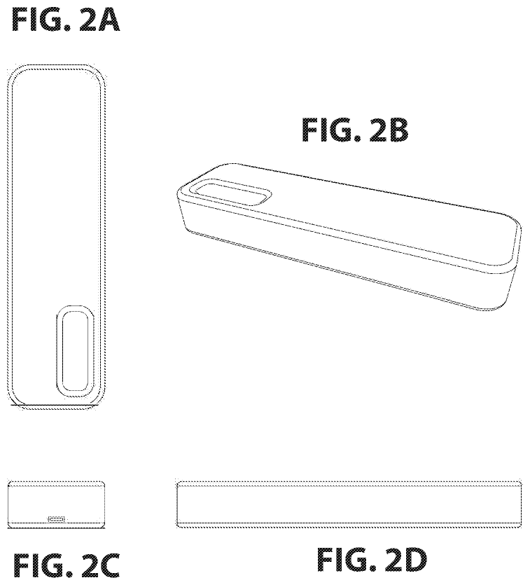

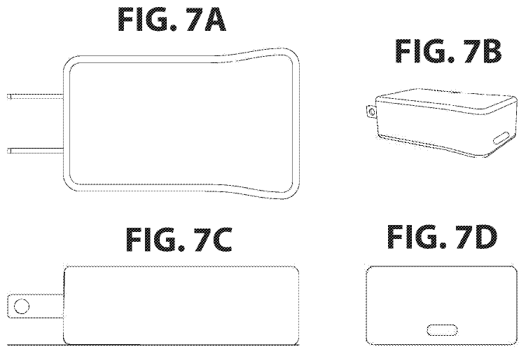

According to an embodiment, the control unit 105 is configured to power a suction mechanism (e.g., an electronic vacuum pump) which is configured to remove air from the tube section 110, thus creating suction within the tube section 110 during use. According to an embodiment, the suction mechanism is coupled to the control unit 105 and/or housed within the control unit 105. According to an embodiment, the control unit 105 is battery powered. The battery may be removable, fixed, and/or rechargeable. According to an embodiment, the control unit 105 is powered and/or is recharged via a power source such as, e.g., a power converter 130 (as shown in various angles in FIGS. 2A-2D) coupled to a wall plug connection 135. According to an embodiment, the power converter connects to the control unit 105 via a connector port 125. FIGS. 2A-2D show detail of the power converter 130. FIG. 2A is a top view. FIG. 2B is a perspective view. FIG. 2C is a side view. FIG. 2D is a front view. According to an embodiment, the wall plug connection is coupled to the power converter via a connector such as, e.g., a type C cable 145. FIGS. 7A-7D show detail of the plug connection 135. FIG. 7A is a top view. FIG. 7B is a perspective view. FIG. 7C is a side view. FIG. 7D is a rear view.

According to an embodiment, the control unit 105 is removably coupled to the tube section 110 at an upper portion of the tube section 110 via a threaded connector 140 (as shown in FIGS. 3B-3D). It is noted, however, that the control unit 105 may be coupled to the tube section 110 via any suitable connector type.

According to an embodiment, the tube section 110 includes a transparent tube 150. The transparent tube 150 may be clear acrylic, plastic, and/or any other suitable material. According to an embodiment, the transparent tube 150 is configured to receive a human penis and is transparent, enabling a user to view the penis within the tube 150.

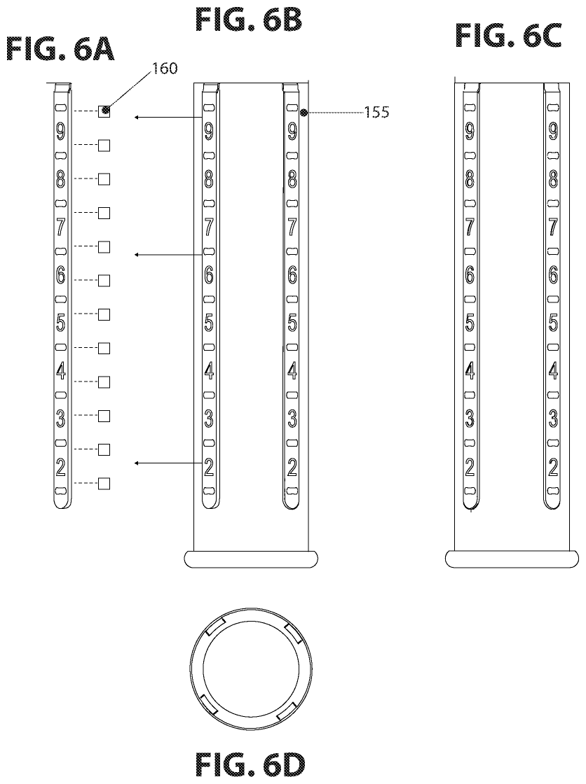

According to an embodiment, the tube section 110 further includes a silicon ruler section 155 configured to enable a user to determine the change in length of the penis during use. FIGS. 6A-6D show detail of the tube section 110. FIG. 6A is a front view of the ruler section 150 having multiple light sources 160. FIG. 6B is a side view of the tube section 110. FIG. 6C is a front view of the tube section 110. FIG. 6D is a bottom view of the tube section 110.

According to an embodiment, the tube section further includes a plurality of light sources 160 (shown in FIG. 6A), positioned on an inner surface of the tube section 110. According to an embodiment, the light sources 160 are positioned opposite the outer portion of the ruler section 155 (as shown in FIG. 6A). It is noted, however, that the light sources 160 may be positioned at any suitable location to provide light to the penis. In particular, the light sources 160 are configured to provide near infrared (NIR) light wavelengths, ranging from 630 nanometers to 980 nanometers. Light of this wavelength range, in conjunction with a simultaneously applied suction force of the system 100, can further enhance penis growth beyond that of a conventional penis pump. In some embodiments, the light sources 160 are positioned along the length of the tube section 110. In this way, light is applied to both the shaft and head of a penis during use. According to some embodiments of the present invention, the light sources 160 include a plurality of light emitting diodes. It is noted that the NIR light sources 160 of the present system 100 are beneficial and an improvement upon the existing technologies at least due to the facts that infrared technology penetrates deep into tissue and provides consistent heat during the entire session of use.

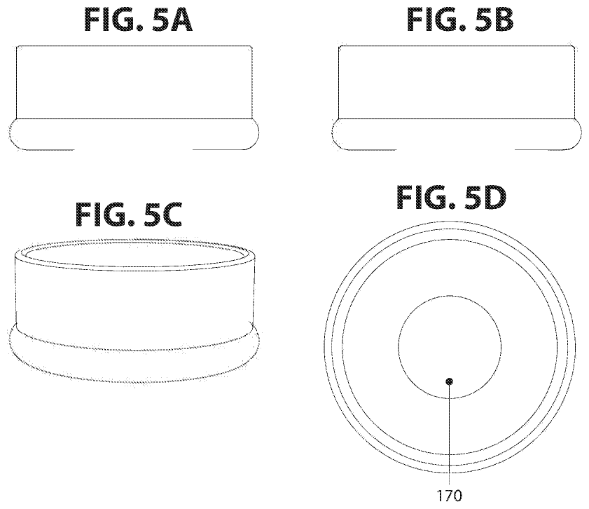

According to an embodiment, a flexible base portion 165 is coupled to the tube portion 110. According to an embodiment, the base portion 165 includes an opening 170 (shown in FIG. 7D) configured to allow the insertion of a human penis through the opening. According to an embodiment, the base portion 165 includes a material (such as, but not limited to, silicone) that is flexible and is capable of providing a seal around the penis enable suction. FIGS. 5A-5D show detail of the base portion 165. FIG. 5A is a front view. FIG. 5B is a side view. FIG. 5C is a perspective view. FIG. 5D is a top view.

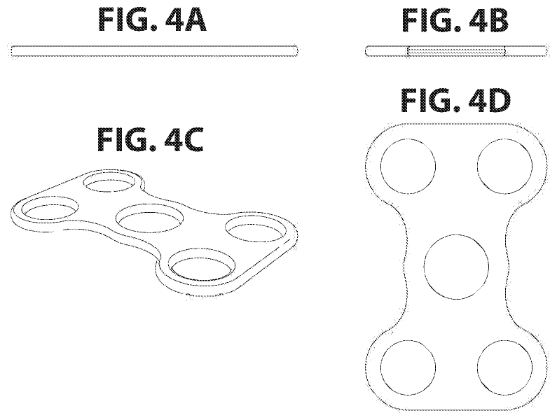

According to an embodiment, the system 100 may further includes a penis strap 175. FIGS. 4A-4D show detail of the penis strap 175. FIG. 4A is a side view. FIG. 4B is a front view. FIG. 4C is a perspective view. FIG. 4D is a top view. Due to the suction provided by the system 100, the penis may gradually be pulled into the tube section 110. The penis strap 175 is configured to wrap around the penis and prevent the testicles from rising into the tube section 110, which may cause discomfort.

As can now be appreciated, disclosed embodiments provide an improved penis pump system 100 for extending a human penis. The apparatus of disclosed embodiments can be conveniently and discretely used for an improved technique for extending a human penis. This provides benefits of improved sexual health and increased confidence for men in a discrete and easy-to-use apparatus.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A penis pump incorporating near infrared light, comprising:
a control unit,
wherein the control unit includes a power source, and
wherein the control unit includes a suction mechanism that is powered by the power source;
a tube section, coupled to the control unit, configured to receive a human penis;
a plurality of light sources housed within the tube section,
wherein the plurality of light sources are configured to direct near infrared light onto the human penis when the human penis is inserted into the tube section; and
a base section configured to provide a seal around the human penis when the human penis is inserted into the tube section.

2. The penis pump as recited in claim 1, wherein the tube section includes a transparent tube, configured to enable visual inspection of the human penis while the human penis is inserted into the transparent tube.

3. The penis pump as recited in claim 1, wherein the plurality of light sources are positioned along a length of the tube section.

4. The penis pump as recited in claim 1, wherein the tube section further includes a ruler section positioned along a length of the tube section.

5. The penis pump as recited in claim 4, wherein the plurality of light sources are coupled to the ruler section.

6. The penis pump as recited in claim 1, wherein the control unit is removably coupled to the tube section.

7. The penis pump as recited in claim 1, wherein the power source is a battery.

8. The penis pump as recited in claim 1, wherein the power source is an external power source.

9. The penis pump as recited in claim 1, wherein the control unit further includes a control panel.

10. The penis pump as recited in claim 9, wherein the control panel includes a plurality of buttons.

11. The penis pump as recited in claim 10, wherein the plurality of buttons are configured to perform functions selected from the group consisting of: powering on the penis pump; powering off the penis pump; increasing pressure within the penis pump; and decreasing pressure within the penis pump.

12. The penis pump as recited in claim 1, wherein the control unit further includes a display configured to display information pertaining to functionalities of the penis pump.

13. The penis pump as recited in claim 1, wherein the base section includes silicone.

14. The penis pump as recited in claim 1, further comprising: a penis strap configured to prevent testicles from rising into the penis pump during use.

15. The penis pump as recited in claim 1, wherein the near infrared light ranges in wavelength from 630 nanometers to 980 nanometers.
